(19) United States
(12) Patent Application Publication
(10) Pub. No.: US 2024/0268995 A1
(43) Pub. Date: Aug. 15, 2024

(54) THERMAL TRANSFER APPARATUS

(71) Applicant: Modular Thermal Technologies, LLC, Pompano Beach, FL (US)

(72) Inventors: Colin M. Hall, Pompano Beach, FL (US); Bradley J. Waugh, Pompano Beach, FL (US); Mark R. Hayes, Pompano Beach, FL (US)

(21) Appl. No.: 18/647,915

(22) Filed: Apr. 26, 2024

Related U.S. Application Data
(63) Continuation-in-part of application No. 18/488,477, filed on Oct. 17, 2023.
Application No. 18/488,477 is a continuation of application No. 29/878,766, filed on Jun. 27, 2023.
(60) Provisional application No. 63/379,969, filed on Oct. 18, 2022.

(51) Int. Cl. A61F 7/10; F25D 3/08

(52) U.S. Cl. CPC A61F 7/10; F25D 3/08; A61F 2007/108; F25D 2303/08222

(57) ABSTRACT

A thermal transfer apparatus and methods of manufacturing and use thereof are provided. The thermal transfer apparatus comprises a first container comprising a first wall, and a second wall connected to the first wall at ends of each wall, thereby defining a first enclosed cavity. At least two protrusions extend from the first wall into the first enclosed cavity towards the second wall and each protrusion comprising a third wall defining a second enclosed cavity. A first thermal transfer media is positioned within the first enclosed cavity. The first thermal transfer media can comprise a first superabsorbent polymer and a first solution at least partially absorbed by the first superabsorbent polymer. A second thermal transfer media is positioned within each second enclosed cavity. The second thermal transfer media can comprise a second superabsorbent polymer and a second solution at least partially absorbed by the second superabsorbent polymer.

### RELATED APPLICATIONS

The present application is a continuation-in-part of U.S. patent application Ser. No. 18/488,477, filed Oct. 17, 2023, which is a continuation of U.S. Design patent application Ser. No. 29/878,766, filed Jun. 27, 2023, and claims priority to U.S. Provisional Patent Application Ser. No. 63/379,969, filed Oct. 18, 2022, each of which is incorporated herein by reference in its entirety.

### FIELD OF USE

The present disclosure relates to a thermal transfer apparatus, a method of use thereof, and a method of manufacture thereof.

### BACKGROUND

Ice packs can be used to temporarily alleviate swelling and/or pain from a portion of a body of a patient. Each different portion of the body may have a different size and shape and the same portion of the body may vary from patient to patient. There are challenges with designing an ice pack suitable for various portions of the body and for various patients.

### SUMMARY

One aspect according to the present disclosure is directed to a thermal transfer apparatus comprising a first container comprising a first wall and a second wall connected to the first wall at ends of each wall, thereby defining a first enclosed cavity. At least two protrusions extend from the first wall into the first enclosed cavity towards the second wall and each protrusion comprises a third wall defining a second enclosed cavity. A first thermal transfer media is positioned within the first enclosed cavity. A second thermal transfer media is positioned within each second enclosed cavity. The first thermal transfer media has a first freezing point lower than a second freezing point of the second thermal transfer media. In various examples, the first thermal transfer media comprises a first superabsorbent polymer and a first solution at least partially absorbed by the first superabsorbent polymer. In various examples, the second thermal transfer media comprises a second superabsorbent polymer and a second solution at least partially absorbed by the second superabsorbent polymer.

It will be understood that the inventions disclosed and described in this specification are not limited to the aspects summarized in this Summary. The reader will appreciate the foregoing details, as well as others, upon considering the following detailed description of various non-limiting and non-exhaustive aspects according to this specification.

### BRIEF DESCRIPTION OF THE DRAWINGS

The features and advantages of the examples presented herein, and the manner of attaining them, will become more apparent, and the examples will be better understood, by reference to the following description taken in conjunction with the accompanying drawings, wherein:

FIG. 1 is top view illustrating an example of a thermal transfer apparatus according to the present disclosure;

FIG. 2A is a cross-section view of the thermal transfer apparatus of FIG. 1 taken across lines 2A-2A;

FIG. 2B is a cross-section view of the thermal transfer apparatus of FIG. 1 taken across lines 2B-2B;

FIG. 3 is top view illustrating an example of a thermal transfer apparatus comprising two modules according to the present disclosure;

FIG. 4 is top view illustrating an example of a thermal transfer apparatus comprising four modules according to the present disclosure;

FIG. 5 is top view illustrating an example of a thermal transfer apparatus comprising four modules configured in a T-shape according to the present disclosure;

FIG. 6 is a front perspective view illustrating an example of a thermal transfer apparatus according to the present disclosure;

FIG. 7 is a front elevational view of the thermal transfer apparatus of FIG. 6;

FIG. 8 is a left side elevational view of the thermal transfer apparatus of FIG. 6;

FIG. 9 is a right side elevational view of the thermal transfer apparatus of FIG. 6;

FIG. 10 is a rear elevational view of the thermal transfer apparatus of FIG. 6;

FIG. 11 is a top view of the thermal transfer apparatus of FIG. 6;

FIG. 12 is a bottom view of the thermal transfer apparatus of FIG. 6;

FIG. 13 is a cross-sectional view of the thermal transfer apparatus of FIG. 6, sectioned along line 13-13 in FIG. 11;

FIG. 14 is a cross-sectional view of the thermal transfer apparatus of FIG. 6, sectioned along line 14-14 in FIG. 11;

FIG. 15 is a cross-sectional view of the thermal transfer apparatus of FIG. 6, sectioned along line 15-15 in FIG. 11;

FIG. 16 is a cross-sectional view of the thermal transfer apparatus of FIG. 6, sectioned along line 16-16 in FIG. 11;

FIG. 17 is a front perspective view of an example of a thermal transfer apparatus comprising two modules according to the present disclosure;

FIG. 18 is a top view of the thermal transfer apparatus of FIG. 17 shown in a first configuration of use;

FIG. 19 is a bottom view of the thermal transfer apparatus of FIG. 17;

FIG. 20 is a front perspective view illustrating an example of a thermal transfer apparatus comprising four modules according to the present disclosure;

FIG. 21 is a top view of the thermal transfer apparatus of FIG. 20;

FIG. 22 is a bottom view of the thermal transfer apparatus of FIG. 20;

FIG. 23 is a front perspective view illustrating an example of a thermal transfer apparatus comprising four modules configured in a T-shape according to the present disclosure;

FIG. 24 is a top view of the thermal transfer apparatus of FIG. 23;

FIG. 25 is a bottom view of the thermal transfer apparatus of FIG. 23; and

FIG. 26 is a chart illustrating a temperature profile of several experimental examples of the thermal transfer apparatus of FIG. 1.

Corresponding reference characters indicate corresponding parts throughout the several views. The exemplifications set out herein illustrate certain embodiments, in one form, and such exemplifications are not to be construed as limiting the scope of the appended claims in any manner.

### DESCRIPTION

Various examples are described and illustrated herein to provide an overall understanding of the structure, function, and use of the disclosed transfer apparatus and methods of using and manufacturing the transfer apparatus. The various examples described and illustrated herein are non-limiting and non-exhaustive. Thus, the invention is not limited by the description of the various non-limiting and non-exhaustive examples disclosed herein. Rather, the invention is defined solely by the claims. The features and characteristics illustrated and/or described in connection with various examples may be combined with the features and characteristics of other examples. Such modifications and variations are intended to be included within the scope of this specification. As such, the claims may be amended to recite any features or characteristics expressly or inherently described in, or otherwise expressly or inherently supported by, this specification. Further, Applicant reserves the right to amend the claims to affirmatively disclaim features or characteristics that may be present in the prior art. The various examples disclosed and described in this specification can comprise, consist of, or consist essentially of the features and characteristics as variously described herein.

Any references herein to “various examples”, “some examples”, “one example”, “an example”, “non-limiting examples”, or like phrases mean that a particular feature, structure, or characteristic described in connection with the example is included in at least one example. Thus, appearances of the phrases “in various examples”, “in some examples”, “in one example”, “in an example”, “in a non-limiting example”, or like phrases in the specification do not necessarily refer to the same example. Furthermore, the particular described features, structures, or characteristics may be combined in any suitable manner in an example or examples. Thus, the particular features, structures, or characteristics illustrated or described in connection with one example may be combined, in whole or in part, with the features, structures, or characteristics of another example or other examples without limitation. Such modifications and variations are intended to be included within the scope of the present examples.

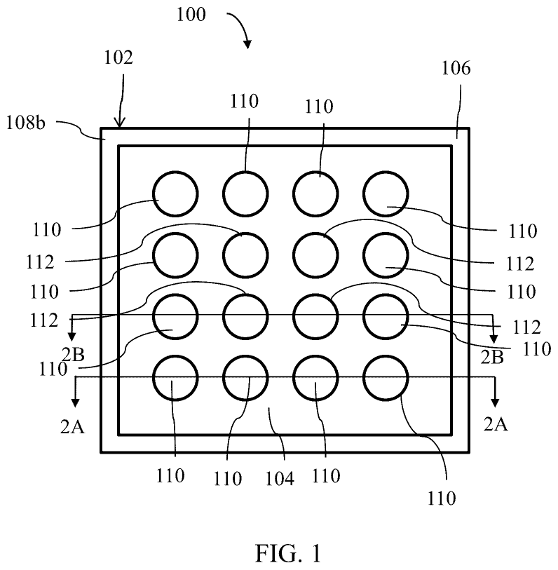

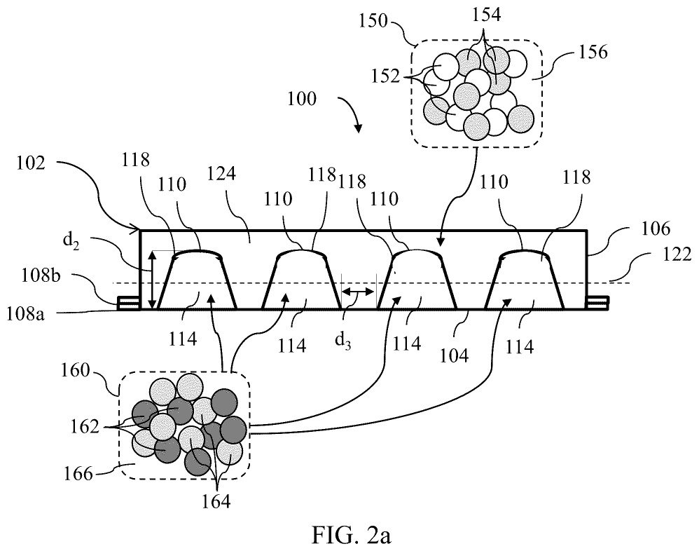

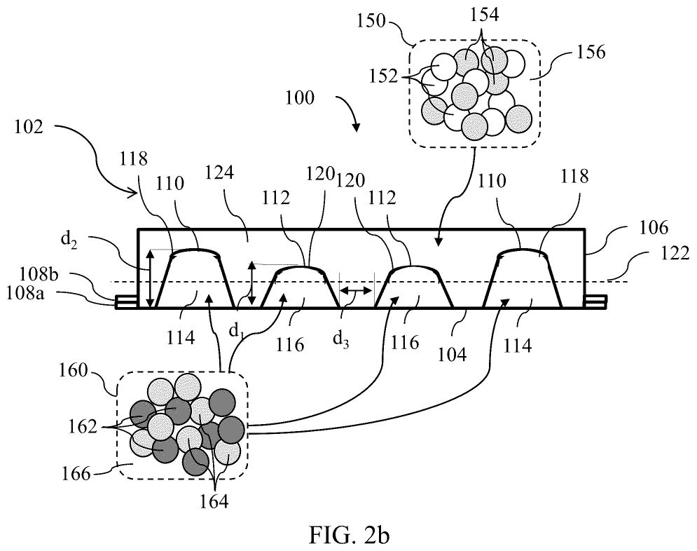

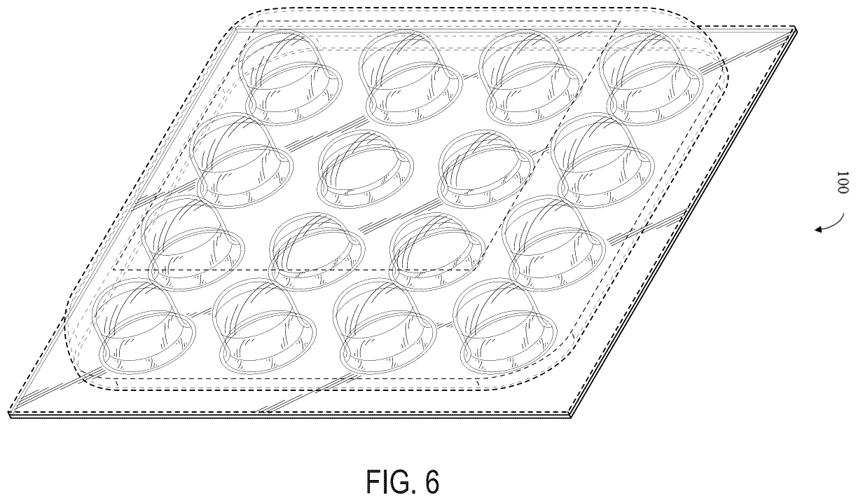

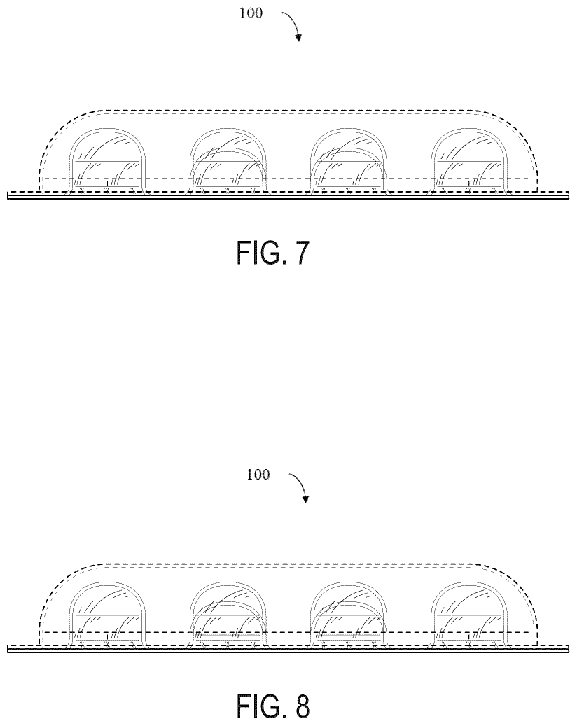

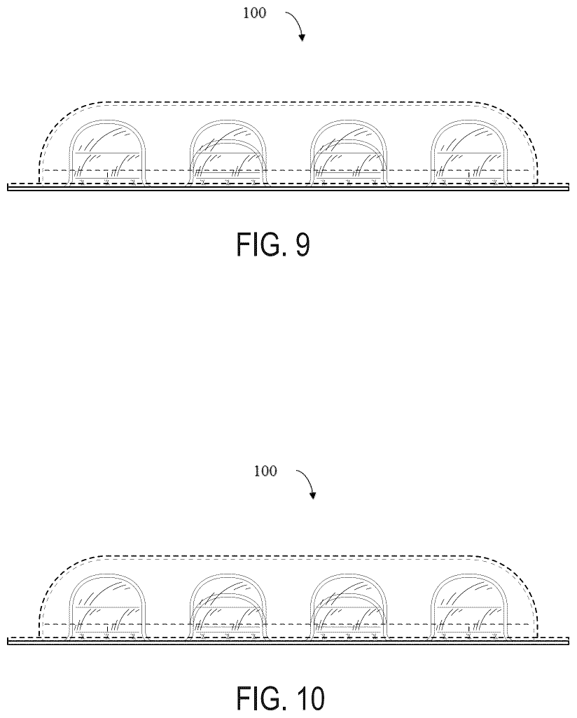

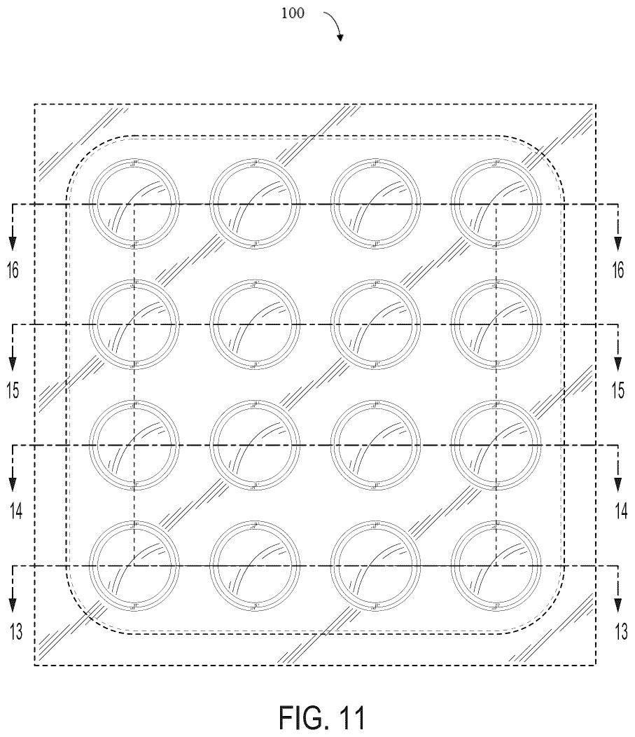

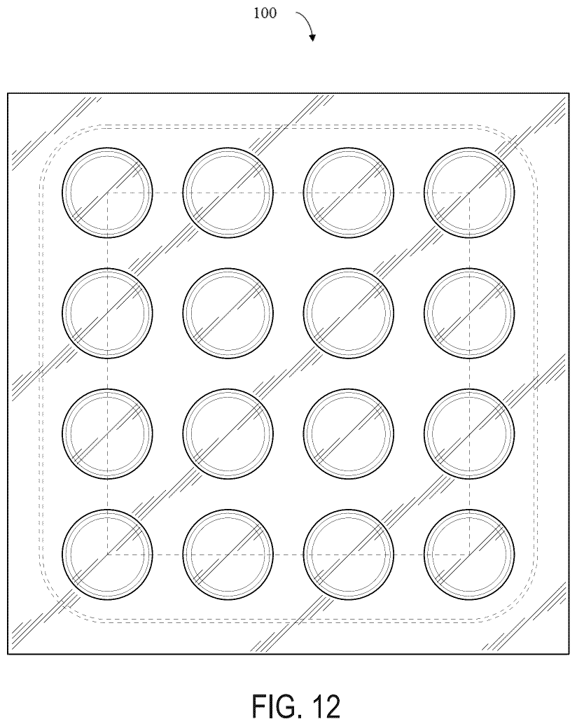

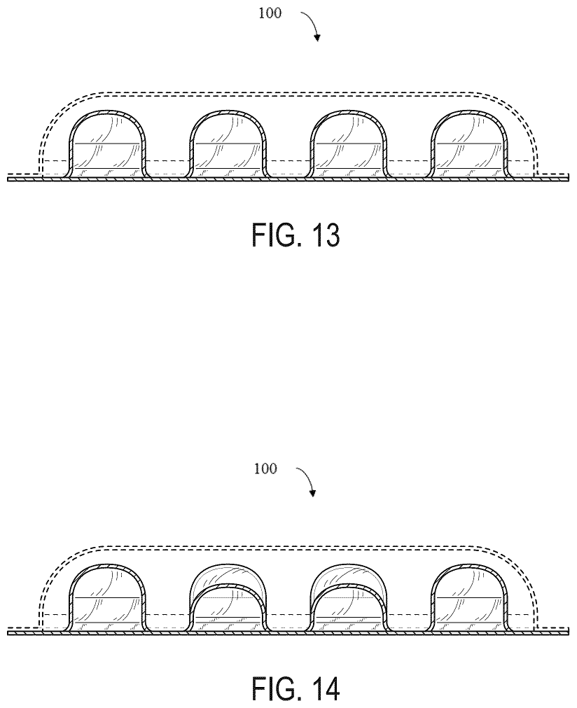

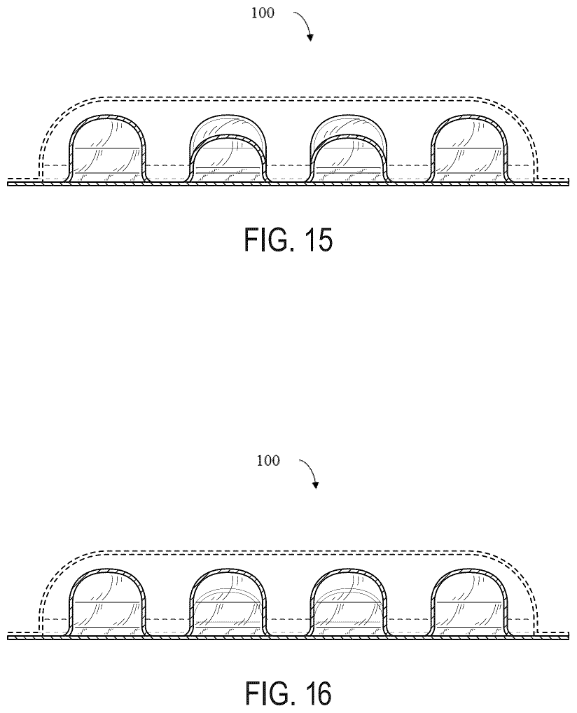

Referring to FIGS. 1 and 2A-2B, a thermal transfer apparatus 100 is provided. The thermal transfer apparatus 100 comprises a first container 102 and at least four protrusions, such as, for example, 16 protrusions as shown in FIG. 1 or another number of protrusions (not shown). The first container 102 comprises a first wall 104 and a second wall 106. The second wall 106 is connected to the first wall 104, thereby defining a first enclosed cavity 124. For example, the second wall 106 can comprise edges 108b and the first wall 104 can comprise edges 108a. The edges 108a and 108b can be fused together by, for example, heat sealing or other adhesive technique such that the first wall 104 is fused to the second wall 106. An example of the thermal transfer apparatus 100 is shown in FIGS. 6-16.

Referring back to FIGS. 1 and 2A-2B, the at least four protrusions can comprise protrusions 110 and 112. The protrusions 110 and 112 extend from the first wall 104 into the first enclosed cavity 124 towards the second wall 106. For example, the protrusions 110 and 112 can be integral with the first wall 104.

As illustrated in FIG. 1, the protrusions 110 and 112 can comprise a rounded cross-section in a plane (e.g., plane 122 in FIG. 2, which is extending out of the page) parallel to the first wall 104. In various examples, the protrusions 110 and 112 can comprise a different shape. Referring to FIGS. 2A-2B, each protrusion 110 and 112 can taper away from the first wall 104.

For example, a diameter of the rounded cross-section of each protrusion 110 and 112 can decrease as one moves away from the first wall 104 towards the second wall 106.

In certain examples, the protrusions 110 and 112 comprise at least two long protrusions and at least two short protrusions, respectively. For example, protrusions 110 can be long protrusions and protrusions 112 can be short protrusions. The short protrusions 112 extend a first distance, d₁, from the first wall 104 into the first enclosed cavity 124 towards the second wall 106. The long protrusions 110 extend a second distance, d₂, from the first wall 104 into the first enclosed cavity 124 towards the second wall 106. The short protrusions 112 are positioned intermediate two or more of the long protrusions 110. In various examples, the long protrusions 110 extend along the perimeter of the thermal transfer apparatus 100 and the short protrusions 112 are in the interior of the thermal transfer apparatus 100 and can be surrounded by the long protrusions 110. The positioning of the long and short protrusions 110 and 112, respectively can enable the thermal transfer apparatus 100 to be more conformal to a portion of a body part it is brought into contact therewith.

In certain examples where the protrusions 110 and 112 comprise at least two long protrusions and at least two short protrusions, respectively, the second distance, d₂, is greater than the first distance, d₁. For example, the second distance is greater than the first distance by at least 0.05 inches, such as, for example, by at least 0.1 inches (2.5 mm), or by at least 0.125 inches (3.175 mm). The first distance, d₁, can be in a range of 0.25 inches (6.35 mm) to 1.25 inches (31.75 mm), such as, for example, 0.5 inches (12.7 mm) to 1 inch (25.4 mm), 0.6 inches (15.2 mm) to 0.95 inches (24.1 mm), or 0.7 inches (17.8 mm) to 0.9 inches (22.9 mm). The second distance, d₂, can be in a range of 0.75 inches (19.1 mm) to 1.5 inches (38.1 mm), such as, for example, 0.8 inches (20.3 mm) to 1.25 inches (31.75 mm), 0.9 inches (22.9 mm) to 1.2 inches (30.5 mm), or 0.95 inches (24.1 mm) to 1.2 inches (30.5 mm).

In certain examples, the protrusions 110 and 112 can comprise substantially the same length. For example, the first distance, d₁, can be substantially the same as the second distance, d₂, and each can be in a range of 0.25 inches (6.35 mm) to 1.5 inches (38.1 mm), such as, for example, 0.5 inches (12.7 mm) to 1 inch (25.4 mm), 0.6 inches (15.2 mm) to 0.95 inches (24.1 mm), 0.7 inches (17.8 mm) to 0.9 inches (22.9 mm), 0.8 inches (20.3 mm) to 1.25 inches (31.75 mm), 0.9 inches (22.9 mm) to 1.2 inches (30.5 mm), or 0.95 inches (24.1 mm) to 1.2 inches (30.5 mm).

In certain examples, the protrusions 110 and 112 can be arranged in a grid, such as, for example, four rows of four columns as illustrated in FIG. 1, five rows of five columns, or eight rows of eight columns, or another configuration. Each protrusion 110 and 112 can be spaced away from an adjacent protrusion a substantially uniform distance. For example, referring to FIGS. 2A-2B, a third distance, d₃, between each protrusion 110 and 112 can be in a range of 0.1 inches to 1.25 inches (31.75 mm), such as, for example, 0.3 inches (7.6 mm) to 1.25 inches (31.75 mm), 0.5 inches (12.7 mm) to 1.1 inches (27.9 mm), or 0.7 inches (17.8 mm) to 1.1 inches (27.9 mm).

Each protrusion 110 and 112 comprises a third wall 118 and 120, respectively, defining a second enclosed cavities 114 and 116, respectively. In various examples, the third walls 118 and 120 are fused to the first wall 104 by, for example, heat sealing. The first wall 104, the second wall 106, and the third walls 118 and 120, each can comprise a thickness in a range of 0.5 mm to 5 mm such that the thermal transfer apparatus 100 can remain pliable. In various examples, the first wall 104, the second wall 106, and the third walls 118 and 120 each can comprise plastic and may be substantially transparent.

A first thermal transfer media 150 is positioned within the first enclosed cavity 124. A second thermal transfer media 160 is positioned within each second enclosed cavity 114 and 116. In certain examples, the first thermal transfer media 150 has a first freezing point lower than a second freezing point of the second thermal transfer media. For example, the first freezing point is lower than the second freezing point by at least 3 degrees Celsius, such as, for example, at least 5 degrees Celsius, or at least 8 degrees Celsius. The difference in freezing points enables the first thermal transfer media 150 to be in a liquid state such that the thermal transfer apparatus is pliable while the second thermal transfer media 160 can be in a solid state. Additionally, maintaining the first thermal transfer media 150 in a liquid state can more efficiently maintain a temperature of the thermal transfer apparatus 100 while applied to a portion of a body of a patient.

The second freezing point can be no lower than temperatures found in conventionally available freezers so that the second thermal transfer media 160 can be frozen in the conventionally available freezers. For example, the second freezing point can be in a range of −5 degrees Celsius to 5 degrees Celsius. The first freezing point can be lower than temperatures found in conventionally available freezers so that the first thermal transfer media 150 may not freeze or the first freezing point may be configured to thaw the first thermal transfer media 150 efficiently at ambient temperature (e.g., 20 degrees Celsius +/−2 degrees Celsius). For example, the first freezing point can be in a range of −15 degrees Celsius to 0 degrees Celsius.

The first thermal transfer media 150 and the second thermal transfer media 160, individually, can comprise a superabsorbent polymer, water, saline, glycol (e.g., propylene glycol), gelatin, a hydrogel, or a combination thereof. For example, the first thermal transfer media 150 and/or the second thermal transfer media 160, individually, can comprise a superabsorbent polymer and a solution (e.g., an aqueous solution) that is at least partially absorbed by the superabsorbent polymer. In certain examples, the first thermal transfer media 150 and the second thermal transfer media 160 can each comprise a different solution that is at least partially absorbed by a corresponding superabsorbent polymer. The ionic concentration of a particular solution affects the superabsorbent polymer's capacity to absorb the solution. Thus, selecting different solutions (e.g., solutions having different ionic concentrations) for use in the first thermal transfer media 150 and the second thermal transfer media 160 can impart different properties to the first thermal transfer media 150 and the second thermal transfer media 160. In various examples, the first thermal transfer media 150, the second thermal transfer media 160, or both the first thermal transfer media 150 and the second thermal transfer media 160 may comprise a solution (e.g., water, a saline solution, a glycol solution) but may not comprise a superabsorbent polymer.

The various superabsorbent polymers discussed herein can be any hydrophilic polymer capable of absorbing an amount of a solution that is equal to or greater than its own mass. For example, a superabsorbent polymer can be a hydrophilic polymer capable of absorbing an amount of a solution that is at least twice its own mass, such as, for example, at least 10 times its own mass, at least 30 times its own mass, at least 50 times its own mass, at least 100 times its own mass, or at least 200 times its own mass. In various examples, a superabsorbent polymer can comprise a polyacrylate, a polyacrylamide, or a copolymer thereof. As another example, a superabsorbent polymer can comprise a maleic anhydride copolymer or an acrylonitrile copolymer. In various examples, the first thermal transfer media 150 and the second thermal transfer media 160 can each comprise the same superabsorbent polymer or polymers. In various examples, the first thermal transfer media 150 and the second thermal transfer media 160 can each comprise a different superabsorbent polymer or different combinations of superabsorbent polymers.

The various solutions discussed herein that are at least partially absorbed by a superabsorbent polymer can comprise water, saline, glycol, or a combination thereof. For example, a solution can comprise distilled, deionized, and/or otherwise purified water. As another example, a solution can comprise a saline solution, such as, for example, a saline solution comprising no greater than 10% salt (e.g., sodium chloride) on a mass-to-volume basis, no greater than 5%, no greater than 2%, or no greater than 1% salt on a mass-to-volume basis. As another example, a solution can consist essentially of glycol (e.g., pure glycol) or can comprise at least 90% glycol by volume, such as, for example, at least 80%, at least 70%, at least 60%, at least 50%, at least 40%, at least 30%, at least 20%, or at least 10% glycol by volume.

The superabsorbent polymers and solutions that are absorbed by the superabsorbent polymers can be in various forms, such as, for example, beads comprising the superabsorbent polymer (e.g., the superabsorbent polymer in bead form) and a solution absorbed by the beads, or a superabsorbent polymer in the form of a ground powder and a solution absorbed by the powder. In some examples, the beads may be substantially spherical. In some examples, the beads may be non-uniform and/or non-spherical in shape. Beads comprising a superabsorbent polymer can be placed in or otherwise exposed to a solution that is absorbable by the superabsorbent polymer. The beads comprising the superabsorbent polymer absorb the solution thereby enlarging the size of the beads. In certain examples, beads comprising the superabsorbent polymer, prior to absorbing the solution, comprises a diameter in a range of 0.25 mm to 5 mm, such as, for example, a diameter in a range 0.5 mm to 4 mm, 0.5 mm to 3 mm, 0.5 mm to 2 mm, 0.5 mm to 1.5 mm, or 0.75 mm to 1.25 mm. As noted above, the ionic concentration of a particular solution affects the superabsorbent polymer's capacity to absorb the solution. Accordingly, beads comprising the superabsorbent polymer can swell to various sizes depending on the ionic concentration of the solution absorbed by the superabsorbent polymer.

As illustrated by the non-limiting aspects of FIGS. 2A-2B, the first thermal transfer media 150 can comprise first beads 152 and second beads 154. The first beads 152 can comprise a first superabsorbent polymer and a first solution absorbed by the first superabsorbent polymer. The second beads 154 can comprise a second superabsorbent polymer and a second solution absorbed by the second superabsorbent polymer. To prepare the first thermal transfer media 150, beads comprising the first superabsorbent polymer can be placed in the first solution and, separately, beads comprising the second superabsorbent polymer can be placed in the second solution. After the first superabsorbent polymer has at least partially absorbed the first solution to produce the first beads 152 and the second superabsorbent polymer has at least partially absorbed the second solution to produce the second beads 154, the first beads 152 and the second beads 154 can be combined to form the first thermal transfer media 150.

In certain examples, the first thermal transfer media 150 comprises a solution 156 that is not absorbed by the first superabsorbent polymer or the second superabsorbent polymer. For example, the solution 156 can comprise at least some of the remaining first solution not absorbed by the first superabsorbent polymer when producing the first beads 152, at least some of the remaining second solution not absorbed by the second superabsorbent polymer when producing the second beads 154, and/or an additional solution that is not the first solution or the second solution.

In certain examples, the first thermal transfer media 150 can comprise the first beads 152 without the second beads 154 and/or without the solution 156. In certain examples, the first thermal transfer media 150 can comprise additional beads that are different from the first beads 152 and the second beads 154 (e.g., beads comprising a superabsorbent polymer and a solution, different from the first solution and the second solution, that is absorbed by the superabsorbent polymer).

In certain examples, a concentration of the first beads 152 and first solution not absorbed by the first beads 152 can make up 100% of the first thermal transfer media 150, by volume. In certain examples, a concentration of the first beads 152 and first solution not absorbed by the first beads 152 can make up less than 100% of the first thermal transfer media 150, by volume, such as, for example, 90%, 80%, 70%, 60%, 50%, 40%, 30%, 20%, or 10% of the first thermal transfer media 150, by volume, with the second beads 154 and second solution not absorbed by the second beads 154 and/or an additional solution making up the remining volume of the first thermal transfer media 150. The relative concentrations of the first beads 152, the second beads 154, and or the solution 156 can be configured to affect various properties of the first thermal transfer media 150, such as, for example, freezing point, thermal transfer efficiency, and/or flexibility.

Referring back to the non-limiting aspect of FIGS. 2A-2B, the second thermal transfer media 160 can comprise third beads 162 and fourth beads 164. The third beads 162 can comprise a third superabsorbent polymer and a third solution absorbed by the third superabsorbent polymer. The fourth beads 164 can comprise a fourth superabsorbent polymer and a fourth solution absorbed by the fourth superabsorbent polymer. To prepare the second thermal transfer media 160, beads comprising the third superabsorbent polymer can be placed in the third solution and, separately, beads comprising the fourth superabsorbent polymer can be placed in the fourth solution. After the third superabsorbent polymer has at least partially absorbed the third solution to produce the third beads 162 and the fourth superabsorbent polymer has at least partially absorbed the fourth solution to produce the fourth beads 164, the third beads 162 and the fourth beads 164 can be combined to form the second thermal transfer media 160.

In certain examples, the second thermal transfer media 160 comprises a solution 166 that is not absorbed by the third superabsorbent polymer or the fourth superabsorbent polymer. For example, the solution 166 can comprise at least some of the remaining third solution not absorbed by the third superabsorbent polymer when producing the third beads 162, at least some of the remaining fourth solution not absorbed by the fourth superabsorbent polymer when producing the fourth beads 164, and/or an additional solution that is not the third solution or the fourth solution.

In certain examples, the second thermal transfer media 160 can comprise the third beads 162 without the fourth beads 164 and/or without the solution 166. In certain examples, the second thermal transfer media 160 can comprise additional beads that are different from the third beads 162 and the fourth beads 164 (e.g., beads comprising a superabsorbent polymer and a solution, different from the third solution and the fourth solution, that is absorbed by the superabsorbent polymer).

In certain examples, a concentration of the third beads 162 and third solution not absorbed by the third beads 162 can make up 100% of the second thermal transfer media 160, by volume. In certain examples, a concentration of the third beads 162 and third solution not absorbed by the third beads 162 can make up less than 100% of the second thermal transfer media 160, by volume, such as, for example, 90%, 80%, 70%, 60%, 50%, 40%, 30%, 20%, or 10% of the second thermal transfer media 160, by volume, with the fourth beads 164 and fourth solution not absorbed by the fourth beads 164 and/or an additional solution making up the remining volume of the second thermal transfer media 160. The relative concentrations of the third beads 162, the fourth beads 164, and or the solution 166 can be configured to affect various properties of the second thermal transfer media 160, such as, for example, freezing point, thermal transfer efficiency, and/or flexibility.

In various examples, the first thermal transfer media 150 and/or second thermal transfer media 160 can comprise gelatin-based hydrogels, such as, for example, those as defined in “Sustainable and Reusable Gelatin-Based Hydrogel ‘Jelly Ice Cubes’ as Food Coolant. I: Feasibilities and Challenges” by Zou et al. ACS Sustainable Chem. Eng. 2021, 9, 15357-15364 and “Sustainable and Reusable Gelatin-Based Hydrogel ‘Jelly Ice Cubes’ as Food Coolant. II: Ideal Freeze - Thaw Conditions” by Zou et al. ACS Sustainable Chem. Eng. 2021, 9, 15365-15374, both hereby incorporated by reference.

In various examples, the first thermal transfer media 150 and/or the second thermal transfer media 160, individually, can further comprise an antimicrobial agent. In various examples, the walls of first enclosed cavity 124 and/or the walls of the second enclosed cavities 114 and 116 can be coated with or can otherwise comprise an antimicrobial agent. The antimicrobial agent can inhibit bacterial and/or fungal growth in the thermal transfer media. The bacterial and/or fungal growth may not be aesthetically desirable and/or unsanitary. In various examples, the first thermal transfer media 150 and/or second thermal transfer media 160 can comprise a range of 0.01% by weight to 2% by weight of the antimicrobial agent, such as, for example, 0.1% by weight to 2% by weight of the antimicrobial agent, 0.1% by weight to 1% by weight of the antimicrobial agent 0.2% by weight to 0.9% by weight of the antimicrobial agent. In various examples, the antimicrobial agent can comprise silver.

The first thermal transfer media 150 and/or second thermal transfer media 160 can further comprise a dye. For example, the first beads 152 can be a first color (e.g., red), the second beads 154 can be a second color (e.g., clear), the third beads 162 can be a third color (e.g., blue), and the fourth beads 164 can be a fourth color (e.g., green).

A method of removing heat with the thermal transfer apparatus 100 is also provided herein. The method comprises disposing the thermal transfer apparatus 100 in a freezer to cool the thermal transfer apparatus 100 to a temperature of less than or equal to the second freezing point. The second thermal transfer media 160 can freeze and the first thermal transfer media 150 may not freeze depending on the first freezing point and the temperature of the freezer. The thermal transfer apparatus 100 can be removed from the freezer and disposed against a portion of a body of a patient and conformed to the portion of the body of the patient so that the thermal transfer apparatus 100 can absorb heat from the portion of the body of the patient. Heat can be absorbed through the first wall 104 and/or the second wall 106 by the transfer of kinetic energy from particle to particle. When heat is absorbed by the first thermal transfer media 150, the first thermal transfer media 150 may have a non-uniform temperature (e.g., first thermal transfer media 150 closer to the first wall 104 and/or second wall 106 may be warmer than first thermal transfer media 150 further away from the first wall 104 and/or the second wall 106). If the first thermal transfer media 150 has a varying temperature, the density also varies causing circulation and movement of liquid portions of the first thermal transfer media 150. The warmed first thermal transfer media 150 can pass by and contact the third wall 118 and/or 120 and heat can be absorbed there through into the second thermal transfer media 160.

A method of manufacturing the thermal transfer apparatus 100 is also provided herein. The method comprises forming a first plastic sheet to define a first open cavity and at least partially filling the first open cavity with the first thermal transfer media 150. The first plastic sheet corresponds to the second wall 106. The method further comprises separately forming a second plastic sheet to define the protrusions 110 and 112 and a second open cavity within each of the protrusions 110 and 112. The second plastic sheet corresponds to the third walls 118 and 120 of the protrusions 110 and 112, respectively. The method further comprises filling each second open cavity with the second thermal transfer media 160 and connecting (e.g., fusing) a third plastic sheet to at least a portion of the second plastic sheet to enclose each second open cavity, thereby forming the second enclosed cavities 114 and 116. The third plastic sheet corresponds to the first wall 104. The method further comprises positioning the connected second and third plastic sheets such that the protrusions 110 and 112 extend into the first open cavity filled with the first thermal transfer media 150 and connecting (e.g., fusing) the first plastic sheet and the third plastic sheet to enclose the first open cavity, thereby forming the first enclosed cavity 124.

In various examples, the method of manufacturing the thermal transfer apparatus 100 can include preparing the first thermal transfer media 150 and/or the second thermal transfer media 160. Preparing the first thermal transfer media 150 can include exposing a first superabsorbent polymer to a first solution such that the first solution is at least partially absorbed by the first superabsorbent polymer. Preparing the second thermal transfer media 160 can include exposing a second superabsorbent polymer to a second solution such that the second solution is at least partially absorbed by the second superabsorbent polymer.

In some examples, preparing the first thermal transfer media 150 can further include exposing a third superabsorbent polymer to a third solution such that the third solution is at least partially absorbed by the third superabsorbent polymer. The third superabsorbent polymer and third solution at least partially absorbed by the third superabsorbent polymer can be combined with the first superabsorbent polymer and first solution at least partially absorbed by the first superabsorbent polymer.

In some examples, preparing the second thermal transfer media 160 can further include exposing a fourth superabsorbent polymer to a fourth solution such that the fourth solution is at least partially absorbed by the fourth superabsorbent polymer. The fourth superabsorbent polymer and fourth solution at least partially absorbed by the fourth superabsorbent polymer can be combined with the second superabsorbent polymer and second solution at least partially absorbed by the second superabsorbent polymer.

In some examples, the first thermal transfer media 150 can be prepared prior to at least partially filling the first open cavity with the first thermal transfer media 150. In some examples, the second thermal transfer media 160 can be prepared prior to filling each second open cavity with the second thermal transfer media 160.

In some examples, at least partially filling the first open cavity with the first thermal transfer media 150 can comprise preparing the first thermal transfer media 150 in the first open cavity. In some examples, filling each second open cavity with the second thermal transfer media 160 can comprise preparing the second thermal transfer media 160 in each second open cavity.

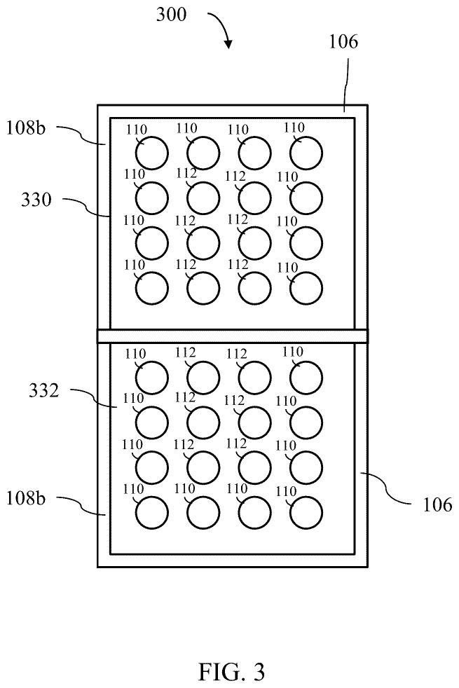

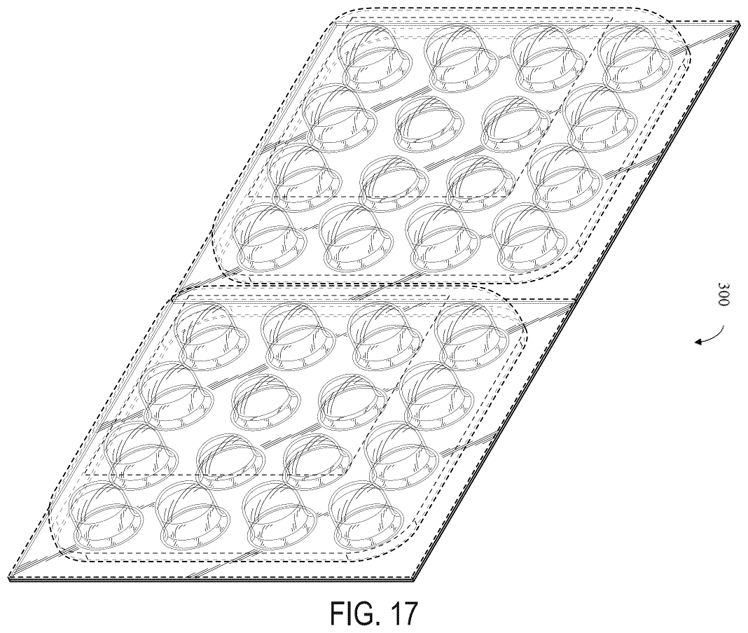

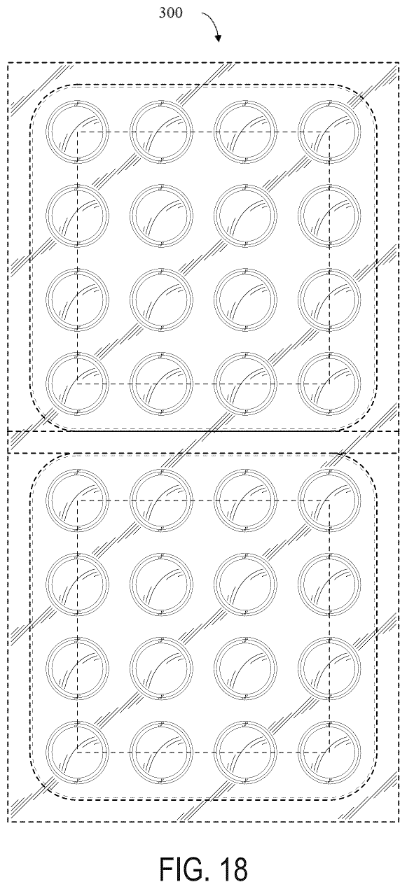

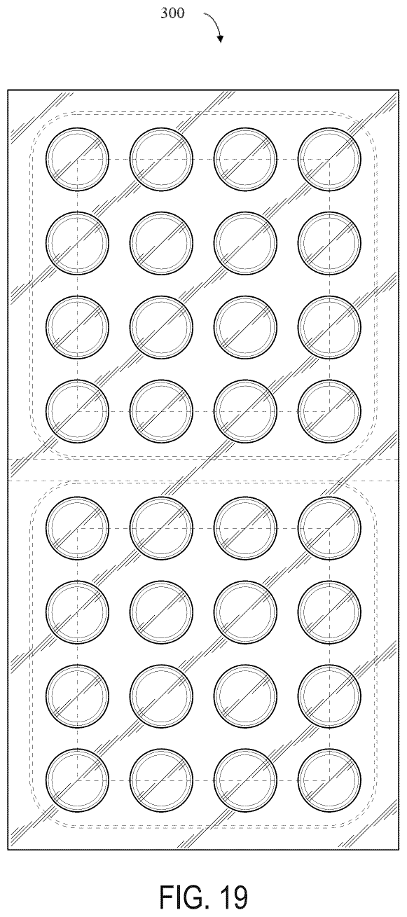

The thermal transfer apparatus can comprise at least two modules. For example, referring to FIG. 3, a thermal transfer apparatus 300 is provided with two modules 330 and 332. Each module 330 and 332 can be the same or different. For example, the modules 330 and 332 can be configured the same as the thermal transfer apparatus 100. The modules are connected together at edges 108a and 108b. In various examples, the first walls 104 of modules 330 and 332 can be part of a single continuous piece through all modules 330 and 332 and the second walls 106 of modules 330 and 332 can be part of a single continuous piece through all modules 330 and 332. The edges 108a and 108b can be portions of the thermal transfer apparatus 300 that are fused together by, for example, a heat sealing or other adhesive technique such that the first wall 104 is fused to the second wall 106. An example of the thermal transfer apparatus 300 is shown in FIGS. 17-19.

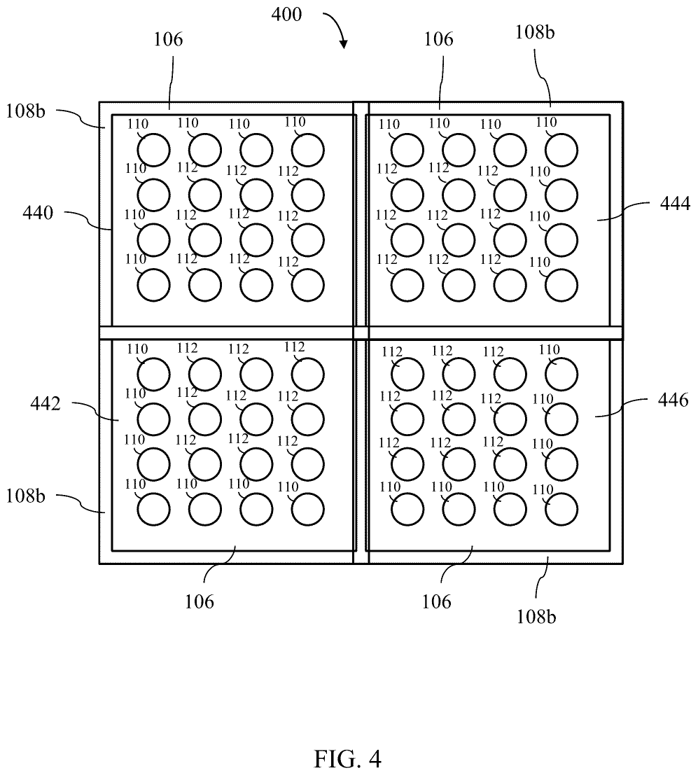

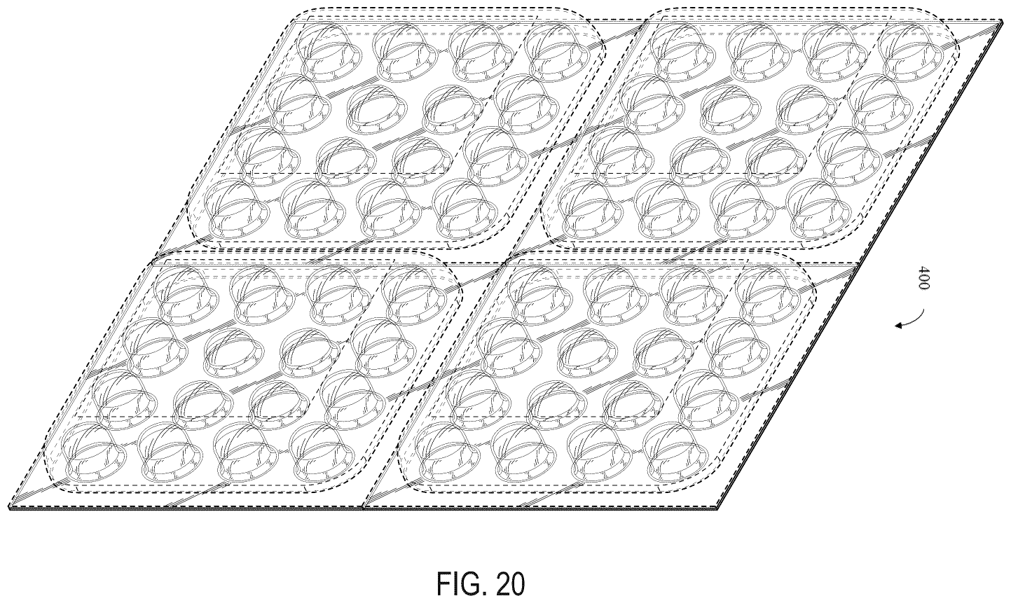

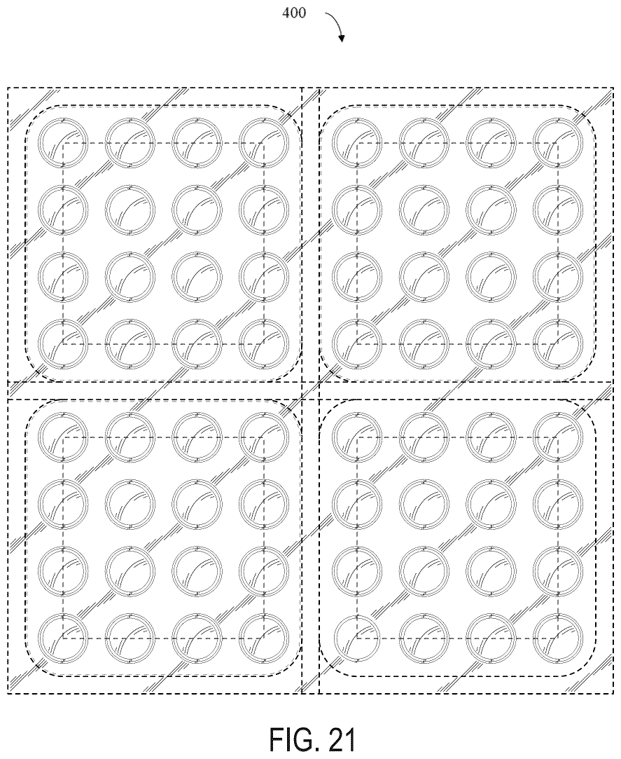

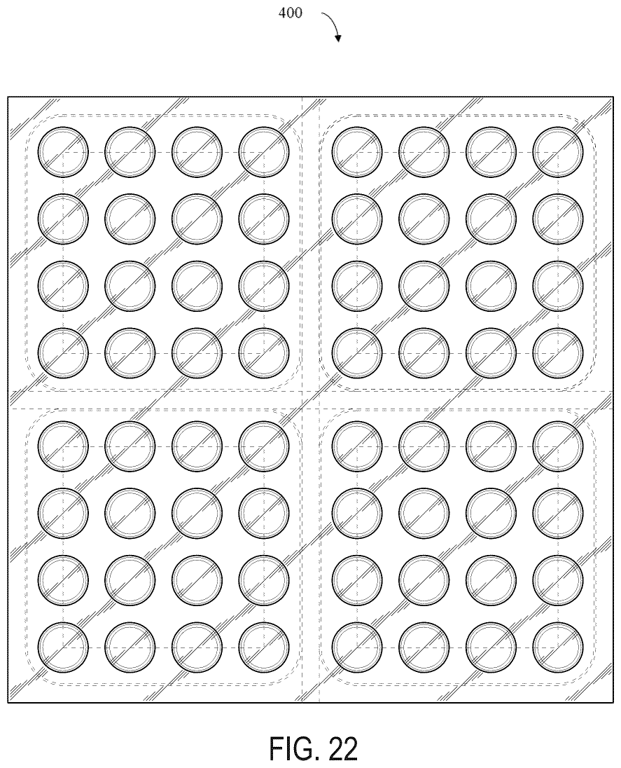

Referring to FIG. 4, a thermal transfer apparatus 400 is provided with four modules 440, 442, 444, and 446 in a first configuration. Each module 440, 442, 444, and 446 can be the same or different. For example, the modules 440, 442, 444, and 446 can be configured the same as the thermal transfer apparatus 100. The modules 440, 442, 444, and 446 are connected together at edges 108a and 108b. In various examples, the first walls 104 of modules 440, 442, 444, and 446 can be part of a single continuous piece through all modules 440, 442, 444, and 446 and the second walls 106 of modules 440, 442, 444, and 446 can be part of a single continuous piece through all modules 440, 442, 444, and 446. The edges 108a and 108b can be portions of the thermal transfer apparatus 400 that are fused together by, for example, a heat sealer such that the first wall 104 is fused to the second wall 106. An example of the thermal transfer apparatus 400 is shown in FIGS. 20-22.

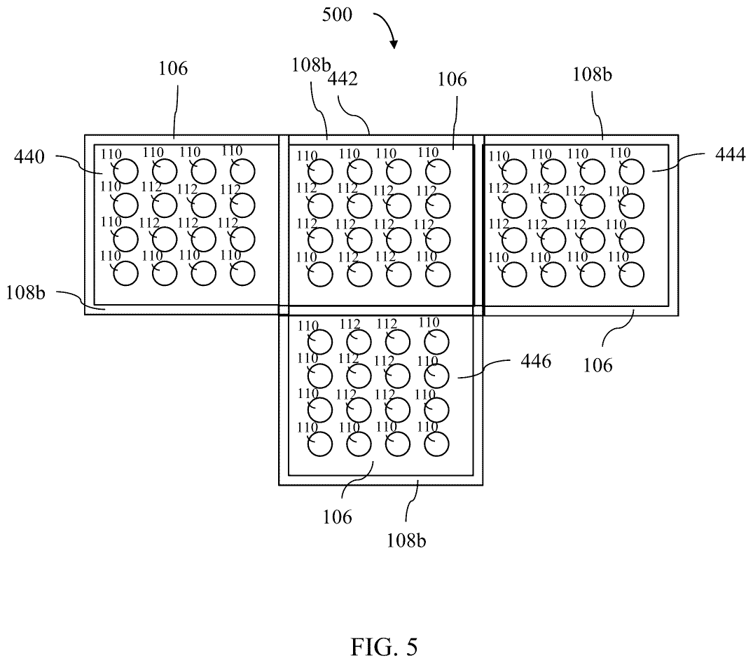

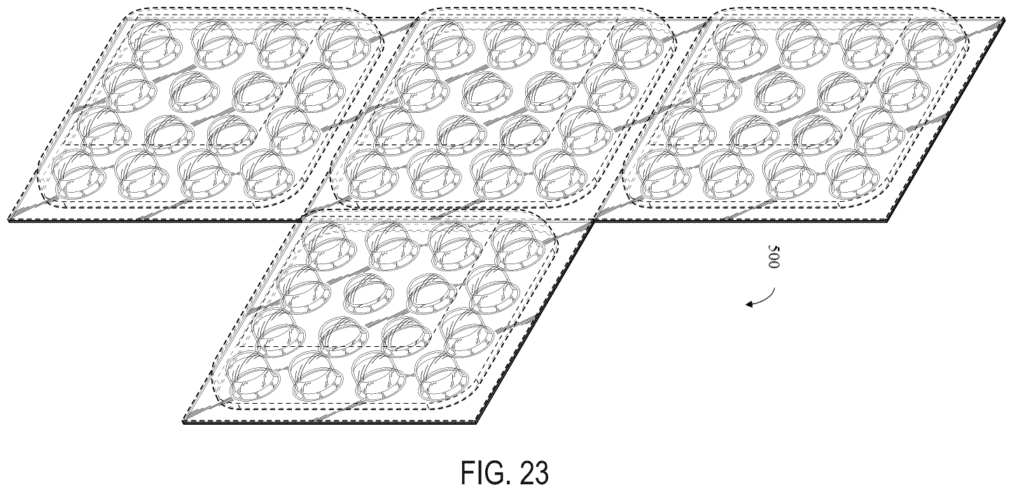

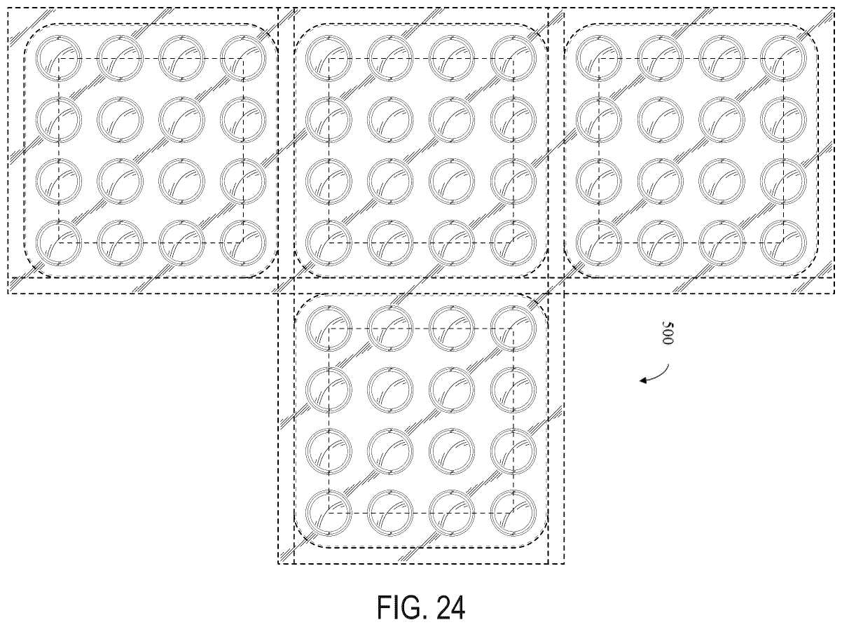

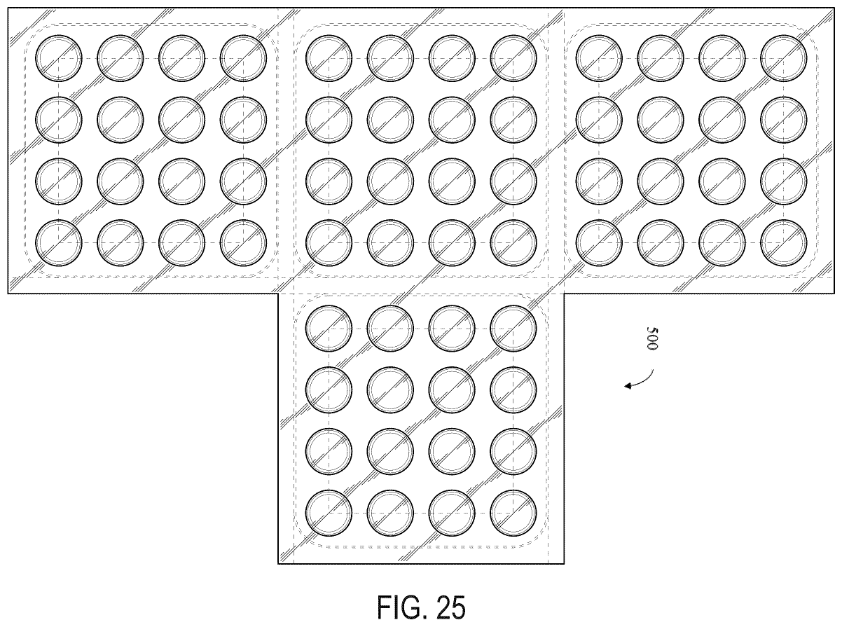

Referring to FIG. 5, a thermal transfer apparatus 500 is provided with modules 440, 442, 444, and 446 in a second configuration, such as, for example, a T-shape. An example of the thermal transfer apparatus 500 is shown in FIGS. 23-25.

### EXAMPLES

Various non-limiting experimental examples (samples) of the thermal transfer apparatus 100 were constructed and tested. The samples of the thermal transfer apparatus 100 each included one first enclosed cavity 124 filled with a first thermal transfer media 150 and thirty-two second enclosed cavities 114 and 116 corresponding to protrusions 110 and 112, respectively. The first enclosed cavity 124 comprised a rectangular shape measuring 6 inches wide by 11.5 inches (29.2 cm) long by 1 inch (2.5 cm) deep. Each of the second enclosed cavities 114 and 116 comprised a cylindrical shape measuring 1 inch (2.5 cm) in diameter and 1 inch (2.5 cm) deep. The second enclosed cavities 114 and 116 were arranged in two, side-by-side square arrays of sixteen (16) cavities.

Each of the samples of the thermal transfer apparatus 100 comprised different compositions of the first thermal transfer media 150 and/or the second thermal transfer media 160, as summarized by Table 1 below. In Table 1, the composition of each first thermal transfer media 150 is summarized in terms of (i) the concentration of first beads 152 and first solution partially absorbed by the first beads 152, (ii) the concentration of second beads 154 and second solution partially absorbed by the second beads 154, if included, and (iii) the concentration of any additional solution 156, if included, in terms a percentage of the total volume of the first thermal transfer media 150. Similarly, the composition of each second thermal transfer media 160 is summarized in terms of (i) the concentration of third beads 162 and third solution partially absorbed by the third beads 162, if included, (ii) the concentration of fourth beads 164 and fourth solution partially absorbed by the fourth beads 164, if included, and (iii) the concentration of any additional solution 166, if included, in terms a percentage of the total volume of the second thermal transfer media 160.

Each of Samples 1, 4, 7, 10, 13, and the control sample of the thermal transfer apparatus 100 were tested by placing the sample in a freezer at a temperature of 0 degrees Fahrenheit and, at one hour intervals for a total of seven (7) hours, observing the extent to which the first thermal transfer media 150 froze, observing the extent to which the second thermal transfer media 160 froze, and observing the flexibility of the thermal transfer apparatus 100 by attempting to fold the thermal transfer apparatus 100, as summarized by Table 2 below. A relative scale of 1 to 5 was selected to quantify the flexibility, where a flexibility of 1 corresponds to the first thermal transfer media 150 being liquid like and the thermal transfer apparatus 100 being easy to fold, and a flexibility of 5 corresponds to the thermal transfer apparatus 100 being very stiff and difficult to fold.

TABLE 1

| | Sample 1 | | Sample 4 | | Sample 5 | | Sample 7 | | Sample 10 | |
|---|---|---|---|---|---|---|---|---|---|---|
| | Conc. | | Conc. | | Conc. | | Conc. | | Conc. | |
| | (Vol- | Solution | (Vol- | Solution | (Vol- | Solution | (Vol- | Solution | (Vol- | Solution |
| | ume %) | Content | ume %) | Content | ume %) | Content | ume %) | Content | ume %) | Content |
| First Thermal | | | | | | | | | | |
| Transfer Media 150 | | | | | | | | | | |
| First Beads 152 + | 100 | 30% Glycol | 50 | 5% Saline | 50 | 5% Saline | 50 | Water | 20 | Pure Glycol |
| First Solution | | | | | | | | | | |
| Second Beads 154 + | 0 | N/A | 0 | N/A | 0 | N/A | 0 | N/A | 40 | 5% Saline |
| Second Solution | | | | | | | | | | |
| Solution 156 | 0 | N/A | 50 | 30% Glycol | 50 | 30% Glycol | 50 | 20% Glycol | 40 | 20% Glycol |
| Second Thermal | | | | | | | | | | |
| Transfer Media 160 | | | | | | | | | | |
| Third Beads 162 + | 0 | N/A | 0 | N/A | 100 | Water | 0 | N/A | 0 | N/A |
| First Solution | | | | | | | | | | |
| Fourth Beads 164 + | 0 | N/A | 0 | N/A | 0 | N/A | 0 | N/A | 0 | N/A |
| Second Solution | | | | | | | | | | |
| Solution 166 | 100 | 5% Saline | 100 | 5% Saline | 0 | N/A | 100 | 5% Saline | 100 | 5% Saline |

| | Sample 13 | | Sample 15 | | Sample 19 | | Control Sample | |
|---|---|---|---|---|---|---|---|---|
| | Conc. | | Conc | | Conc. | | Conc. | |
| | (Vol- | Solution | (Vol- | Solution | (Vol- | Solution | (Vol- | Solution |
| | ume %) | Content | ume %) | Content | ume %) | Content | ume %) | Content |
| First thermal transfer Media 150 | | | | | | | | |
| First Beads 152 + First Solution | 50 | Pure Glycol | 30 | Pure Glycol | 50 | 50% Glycol | 0 | N/A |
| Second Beads 154 + Second Solution | 50 | Water | 70 | Water | 50 | Water | 0 | N/A |
| Solution 156 | 0 | N/A | 0 | N/A | 0 | N/A | 100 | 20% Glycol |
| Second Thermal Transfer Media 160 | | | | | | | | |
| Third Beads 162 + First Solution | 100 | 2% Saline | 100 | 2% Saline | 100 | Water | 0 | N/A |
| Fourth Beads 164 + Second Solution | 0 | N/A | 0 | N/A | 0 | N/A | 0 | N/A |
| Solution 166 | 0 | N/A | 0 | N/A | 0 | N/A | 100 | Water |

TABLE 2

| | Sample 1 | | | Sample 4 | | | Sample 7 | | |
|---|---|---|---|---|---|---|---|---|---|
| | First | Second | | First | Second | | First | Second | |
| Time | Media | Media | | Media | Media | | Media | Media | |
| (Hrs.) | 150 | 160 | Flex. | 150 | 160 | Flex. | 150 | 160 | Flex. |
| 1 | Semi-thick | No | 1 | No | No | 1 | Thick liquid, | No | 1 |
| | liquid, | freeze | | freeze | freeze | | some frost | freeze | |
| | no freeze | | | | | | inside | | |
| 2 | Semi-thick | No | 1 | Semi-thick | No | 1 | Very thick | No | 2 |
| | liquid, | freeze | | liquid, | freeze | | liquid, | freeze | |
| | no freeze | | | no freeze | | | frost inside, | | |
| | | | | | | | no freeze | | |
| 3 | Semi-thick | No | 1 | Semi-thick | 12.5% | 1 | Very thick | 15.625% | 3 |
| | liquid, | freeze | | liquid, | semi-frozen | | liquid, | semi-frozen | |
| | no freeze | | | no freeze | | | 50% | | |
| | | | | | | | hard frost | | |
| | | | | | | | inside | | |
| 4 | Semi-thick | 6.25% | 1 | Semi-thick | 12.5% | 1 | 50% | 30% | 4 |
| | liquid, | semi-frozen | | liquid, | semi-frozen | | frozen | semi-frozen | |
| | no freeze | | | no freeze | | | | | |
| 5 | Thick liquid, | 16% | 2 | Semi-thick | 75% is | 2 | 75% | 47% | 4 |
| | no freeze | semi-frozen | | liquid, | semi-frozen | | frozen | semi-frozen | |
| | | | | no freeze | | | | | |
| 6 | Thick liquid, | 6.25% | 2 | Thick liquid, | 100% | 2 | Middle is | 56% | 4 |
| | frost inside, | frozen, | | frost inside, | semi-frozen | | “mushy,” | frozen, | |
| | no freeze | 15% | | no freeze | | | ends frozen | 44% | |
| | | semi-frozen | | | | | with frost | semi-frozen | |
| | | | | | | | present | | |
| 7 | Thick liquid, | 84% | 2 | Hard | 87.5% | 3 | Middle is | 70% | 5 |
| | frost inside, | frozen | | thick liquid, | frozen, | | “mushy,” | frozen, | |
| | no freeze | | | frost inside, | 12.5% | | ends frozen | 30% | |
| | | | | no freeze | semi-frozen | | with frost | semi-frozen | |
| | | | | | | | present | | |

| | Sample 10 | | | Sample 13 | | | Control Sample | | |
|---|---|---|---|---|---|---|---|---|---|
| | First | Second | | First | Second | | First | Second | |
| Time | Media | Media | | Media | Media | | Media | Media | |
| (Hrs.) | 150 | 160 | Flex. | 150 | 160 | Flex. | 150 | 160 | Flex. |
| 1 | No | No | 1 | Thick stiff | 25% | 1 | No | No | 1 |
| | freeze | freeze | | liquid, | frozen, | | freeze | freeze | |
| | | | | holds the | 40% | | | | |
| | | | | fold | semi-frozen, | | | | |
| | | | | | 15% | | | | |
| | | | | | liquid | | | | |
| 2 | 10% - | No | 1 | Stiff liquidly | 66% | 2 | Gel-like | 18% | 1 |
| | start to | freeze | | matter, | frozen, | | liquid, | frozen, | |
| | freeze | | | holds when | 16% | | temporarily | 82% | |
| | | | | folded | semi-frozen, | | holds when | liquid | |
| | | | | | 16% | | folded | | |
| | | | | | liquid | | | | |
| 3 | 10% - | No | 1 | Gel-like | 78% | 2 | Gel-like | 22% | 2 |
| | start to | freeze | | thick matter, | frozen, | | liquid, | frozen, | |
| | freeze | | | some frost, | 22% | | temporarily | 78% | |
| | | | | holds when | semi-frozen | | holds when | liquid | |
| | | | | folded | | | folded, | | |
| | | | | | | | some frost | | |
| 4 | 10% - | Beads | 1 | Gel-like | 94% | 2.5 | Gel-like | 44% | 2 |
| | start to | semi-frozen | | thick matter, | frozen, | | liquid, | frozen, | |
| | freeze | but still | | flexible, | 6% | | some frost | 56% | |
| | | soft, ice | | holds when | liquid | | | liquid | |
| | | present | | folded | | | | | |
| 5 | 10% - | Beads | 3 | Gel-like | 100% | 2.5 | Liquid is | 66% | 2.5 |
| | start to | semi-frozen | | thick matter, | frozen | | semi-frozen, | frozen, | |
| | freeze | but still | | flexible, | | | flexible, | 34% | |
| | | soft, ice | | but stiff | | | some liquid | liquid | |
| | | present | | | | | moves inside | | |
| 6 | Thick frosty | 100% | 3 | Thick matter, | 100% | 2.5 | Liquid is | 69% | 3 |
| | liquid, | frozen | | very | frozen | | semi-frozen | frozen, | |
| | hard to | | | flexible | | | and flexible, | 31% | |
| | fold | | | but stiff | | | some liquid | liquid | |
| | | | | | | | moves inside | | |
| 7 | Thick frosty | 100% | 3.5 | Very | 100% | 2~2.5 | Flexible | 100% | 3 |
| | liquid, | frozen | | flexible | frozen | | in all | frozen | |
| | hard to | | | in all | | | directions | | |
| | fold | | | directions | | | | | |

As summarized by Table 2, the first thermal transfer media 150 of Sample 13 of the thermal transfer apparatus 100 exhibited flexibility in all directions and the second thermal transfer media 160 was completely frozen after 7 hours. Thus, Sample 13 of the thermal transfer apparatus 100 may be able to efficiently conform the body of a patient, due to the flexible nature of the first thermal transfer media 150, while also providing longer-lasting cooling to the body of the patient.

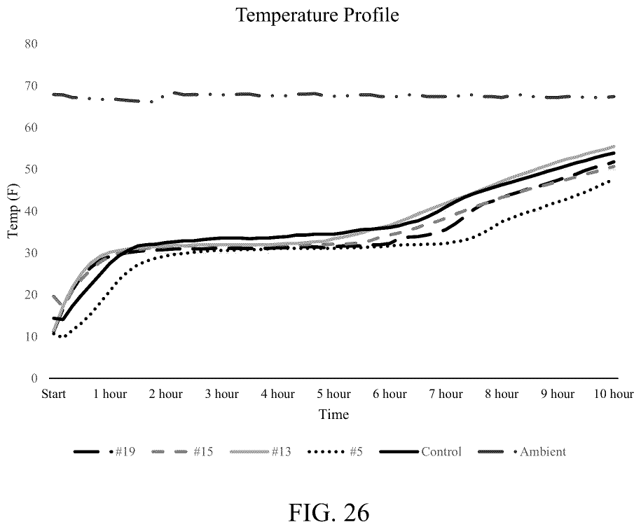

Each of Samples 5, 13, 15, 19, and the control sample of the thermal transfer apparatus 100 were tested by placing the sample in a freezer at a temperature of 0 degrees Fahrenheit, removing the sample from the freezer, and monitoring the temperature of the sample over a 10 hour period while exposed to ambient temperature conditions. FIG. 26 is a chart illustrating the monitored temperature of Samples 5, 13, 15, 19, the control sample, and the ambient temperature over the 10-hour period. As illustrated by FIG. 26, each of the tested samples maintained a temperature of less than 40 degrees Fahrenheit for at least 6.5 hours, with Sample 5 maintaining a temperature of less than 40 degrees Fahrenheit for at least 8.5 hours. Thus, the tested samples of the thermal transfer apparatus 100 may be able to provide cooling to the body of the patient for at least 6.5 hours.

The following numbered clauses are directed to various non-limiting embodiments and aspects according to the present disclosure.

Clause 1. A thermal transfer apparatus comprising: a first container comprising: a first wall, and a second wall connected to the first wall at ends of each wall, thereby defining a first enclosed cavity: at least two protrusions extending from the first wall into the first enclosed cavity towards the second wall and each protrusion comprising a third wall defining a second enclosed cavity: a first thermal transfer media positioned within the first enclosed cavity, wherein the first thermal transfer media comprises a first superabsorbent polymer and a first solution at least partially absorbed by the first superabsorbent polymer: and a second thermal transfer media positioned within each second enclosed cavity, wherein the second thermal transfer media comprises a second superabsorbent polymer and a second solution at least partially absorbed by the second superabsorbent polymer, wherein the first thermal transfer media has a first freezing point lower than a second freezing point of the second thermal transfer media.

Clause 2. The thermal transfer apparatus of Clause 1, wherein: the first thermal transfer media comprises first beads comprising the first superabsorbent polymer and the first solution absorbed by the first superabsorbent polymer, and the second thermal transfer media comprises second beads comprising the second superabsorbent polymer and the second solution absorbed by the second superabsorbent polymer.

Clause 3. The thermal transfer apparatus of any of Clauses 1-2, wherein the first solution comprises a glycol solution.

Clause 4. The thermal transfer apparatus of any of Clauses 1-3, wherein the second solution comprises a saline solution.

Clause 5. The thermal transfer apparatus of any of Clauses 1-4, wherein the first thermal transfer media further comprises a third superabsorbent polymer and a third solution at least partially absorbed by the third superabsorbent polymer.

Clause 6. The thermal transfer apparatus of any of Clauses 1-5, wherein the first thermal transfer media further comprises third beads comprising the third superabsorbent polymer and the third solution absorbed by the third superabsorbent polymer.

Clause 7. The thermal transfer apparatus of Clause 6, wherein the third solution comprises a water solution.

Clause 8. The thermal transfer apparatus of any of Clauses 6-7, wherein the first solution comprises at least 30% glycol by volume.

Clause 9. The thermal transfer apparatus of Clause 8, wherein the first solution consists essentially of glycol.

Clause 10. The thermal transfer apparatus of any of Clauses 6-9, wherein the second solution comprises no greater than 5% salt on a mass-to-volume basis.

Clause 11. The thermal transfer apparatus of Clause 10, wherein the second solution comprises no greater than 2% salt on a mass-to-volume basis.

Clause 12. The thermal transfer apparatus of any of Clauses 6-11, wherein the third solution consists essentially of water.

Clause 13. The thermal transfer apparatus of any of Clauses 1-12, wherein the first superabsorbent polymer and the second superabsorbent polymer, individually, comprises polyacrylate, polyacrylamide, or a copolymer thereof.

Clause 14. The thermal transfer apparatus of any of Clauses 2-13, wherein each of the first beads, prior to absorbing the first solution, and each of the second beads, prior to absorbing the second solution, comprises a diameter in a range of 0.25 mm to 5 mm.

Clause 15. The thermal transfer apparatus of any of Clauses 1-14, wherein the at least two protrusions comprise at least four protrusions are arranged in a grid.

Clause 16. The thermal transfer apparatus of any of Clauses 1-15, further comprising sixteen protrusions including the at least two protrusions, wherein the sixteen protrusions are arranged in four rows and four columns to form a first module.

Clause 17. The thermal transfer apparatus of any of Clauses 1-16, further comprising at least two modules, including the first module, coupled together.

Clause 18. The thermal transfer apparatus of any of Clauses 1-17, wherein: the at least two protrusions comprise at least two short protrusions and at least two long protrusions, the short protrusions extend a first distance from the first wall into the first enclosed cavity towards the second wall, and are positioned intermediate two or more of the long protrusions, and the long protrusions extend a second distance from the first wall into the first enclosed cavity towards the second wall, the second distance is greater than the first distance.

Clause 19. The thermal transfer apparatus of Clause 18, wherein the second distance is greater than the first distance by at least 0.05 inches.

Clause 20. The thermal transfer apparatus of Clause 18, wherein the second distance is greater than the first distance by at least 0.1 inches.

Clause 21. The thermal transfer apparatus of any of Clauses 1-20, wherein the first freezing point is lower than the second freezing point by at least 3 degrees Celsius.

Clause 22. The thermal transfer apparatus of any of Clauses 1-21, wherein the second freezing point is in a range of −5 degrees Celsius to 5 degrees Celsius.

Clause 23. The thermal transfer apparatus of any of Clauses 1-22, wherein the first wall, the second wall, and the third wall, each comprise plastic.

Clause 24. The thermal transfer apparatus of any of Clauses 1-23, wherein the first wall, the second wall, and the third wall, each comprise a thickness in a range of 0.5 mm to 5 mm.

Clause 25. A method comprising: disposing the thermal transfer apparatus of any of Clauses 1-24 in a freezer to cool the thermal transfer apparatus to a temperature of less than or equal to the second freezing point, thereby forming a cooled thermal transfer apparatus: and disposing the cooled thermal transfer apparatus against a portion of a body of a patient and conforming the cooled thermal transfer apparatus against the portion of the body of the patient to absorb heat.

Clause 26. A method of manufacturing the thermal transfer apparatus of any of Clauses 1-24, comprising: forming a first plastic sheet to define a first open cavity, wherein the first plastic sheet corresponds to the second wall: at least partially filling the first open cavity with the first thermal transfer media: forming a second plastic sheet to define the at least two protrusions and a second open cavity within each protrusion, wherein the second plastic sheet corresponds to the third wall of each protrusion: filling each second open cavity with the second thermal transfer media: connecting a third plastic sheet to at least a portion of the second plastic sheet to enclose each second open cavity, thereby forming the second enclosed cavities, wherein the third plastic sheet corresponds to the first wall: positioning the connected second and third plastic sheets such that the protrusions extend into the first open cavity: and connecting the first plastic sheet and the third plastic sheet to enclose the first open cavity, thereby forming the first enclosed cavity.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein, and any minimum numerical limitation recited in this specification is intended to include all higher numerical limitations subsumed therein. Accordingly. Applicant reserves the right to amend this specification, including the claims, to expressly recite any sub-range subsumed within the ranges expressly recited. All such ranges are inherently described in this specification.

The grammatical articles “a”, “an”, and “the”, as used herein, are intended to include “at least one” or “one or more”, unless otherwise indicated, even if “at least one” or “one or more” is expressly used in certain instances. Thus, the foregoing grammatical articles are used herein to refer to one or more than one (i.e., to “at least one”) of the particular identified elements. Further, the use of a singular noun includes the plural, and the use of a plural noun includes the singular, unless the context of the usage requires otherwise.

As used herein, “intermediate” means that the referenced element is disposed between two elements but is not necessarily in contact with those elements. Accordingly, unless stated otherwise herein, an element that is “intermediate” a first element and a second element may or may not be adjacent to or in contact with the first and/or second elements, and other elements may be disposed between the intermediate element and the first and/or second elements.

One skilled in the art will recognize that the herein described thermal support, structures, operations/actions, and objects, and the discussion accompanying them, are used as examples for the sake of conceptual clarity and that various configuration modifications are contemplated. Consequently, as used herein, the specific examples/embodiments set forth and the accompanying discussion are intended to be representative of their more general classes. In general, use of any specific exemplar is intended to be representative of its class, and the non-inclusion of specific components, devices, apparatus, operations/actions, and objects should not be taken as limiting. While the present disclosure provides descriptions of various specific aspects for the purpose of illustrating various aspects of the present disclosure and/or its potential applications, it is understood that variations and modifications will occur to those skilled in the art. Accordingly. the invention or inventions described herein should be understood to be at least as broad as they are claimed and not as more narrowly defined by particular illustrative aspects provided herein.

What is claimed is:

1. A thermal transfer apparatus comprising:
a first container comprising:
a first wall, and
a second wall connected to the first wall at ends of each wall, thereby defining
a first enclosed cavity:
at least two protrusions extending from the first wall into the first enclosed cavity towards the second wall and each protrusion comprising a third wall defining a second enclosed cavity:
a first thermal transfer media positioned within the first enclosed cavity, wherein the first thermal transfer media comprises a first superabsorbent polymer and a first solution at least partially absorbed by the first superabsorbent polymer; and
a second thermal transfer media positioned within each second enclosed cavity, wherein the second thermal transfer media comprises a second superabsorbent polymer and a second solution at least partially absorbed by the second superabsorbent polymer, wherein the first thermal transfer media has a first freezing point lower than a second freezing point of the second thermal transfer media.

2. The thermal transfer apparatus of claim 1, wherein:
the first thermal transfer media comprises first beads comprising the first superabsorbent polymer and the first solution absorbed by the first superabsorbent polymer, and
the second thermal transfer media comprises second beads comprising the second superabsorbent polymer and the second solution absorbed by the second superabsorbent polymer.

3. The thermal transfer apparatus of claim 2, wherein the first solution comprises a glycol solution.

4. The thermal transfer apparatus of claim 3, wherein the second solution comprises a saline solution.

5. The thermal transfer apparatus of claim 4, wherein the first thermal transfer media further comprises a third superabsorbent polymer and a third solution at least partially absorbed by the third superabsorbent polymer.

6. The thermal transfer apparatus of claim 5, wherein the first thermal transfer media further comprises third beads comprising the third superabsorbent polymer and the third solution absorbed by the third superabsorbent polymer.

7. The thermal transfer apparatus of claim 6, wherein the third solution comprises a water solution.

8. The thermal transfer apparatus of claim 7, wherein the first solution comprises at least 30% glycol by volume.

9. The thermal transfer apparatus of claim 8, wherein the first solution consists essentially of glycol.

10. The thermal transfer apparatus of claim 7, wherein the second solution comprises no greater than 5% salt on a mass-to-volume basis.

11. The thermal transfer apparatus of claim 10, wherein the second solution comprises no greater than 2% salt on a mass-to-volume basis.

12. The thermal transfer apparatus of claim 7, wherein the third solution consists essentially of water.

13. The thermal transfer apparatus of claim 2, wherein the first superabsorbent polymer and the second superabsorbent polymer, individually, comprises polyacrylate, polyacrylamide, or a copolymer thereof.

14. The thermal transfer apparatus of claim 2, wherein each of the first beads, prior to absorbing the first solution, and each of the second beads, prior to absorbing the second solution, comprises a diameter in a range of 0.25 mm to 5 mm.

15. The thermal transfer apparatus of claim 1, wherein the at least two protrusions comprise at least four protrusions are arranged in a grid.

16. The thermal transfer apparatus of claim 1, further comprising sixteen protrusions including the at least two protrusions, wherein the sixteen protrusions are arranged in four rows and four columns to form a first module.

17. The thermal transfer apparatus of claim 16, further comprising at least two modules, including the first module, coupled together.

18. The thermal transfer apparatus of claim 1, wherein:
the at least two protrusions comprise at least two short protrusions and at least two long protrusions,
the short protrusions extend a first distance from the first wall into the first enclosed cavity towards the second wall, and are positioned intermediate two or more of the long protrusions, and
the long protrusions extend a second distance from the first wall into the first enclosed cavity towards the second wall, the second distance is greater than the first distance.

19. The thermal transfer apparatus of claim 18, wherein the second distance is greater than the first distance by at least 0.05 inches.

20. The thermal transfer apparatus of claim 18, wherein the second distance is greater than the first distance by at least 0.1 inches.

21. The thermal transfer apparatus of claim 1, wherein the first freezing point is lower than the second freezing point by at least 3 degrees Celsius.

22. The thermal transfer apparatus of claim 1, wherein the second freezing point is in a range of −5 degrees Celsius to 5 degrees Celsius.

23. The thermal transfer apparatus of claim 1, wherein the first wall, the second wall, and the third wall, each comprise plastic.

24. The thermal transfer apparatus of claim 1. wherein the first wall. the second wall. and the third wall. each comprise a thickness in a range of 0.5 mm to 5 mm.
